(19) United States
(12) Patent Application Publication
(10) Pub. No.: US 2005/0251194 A1
(43) Pub. Date: Nov. 10, 2005

(54) Curved wing balloon and manufacture thereof

(75) Inventor: Tom McHale, Furbo (IE)

Correspondence Address: KENYON & KENYON, 1500 K STREET NW, SUITE 700, WASHINGTON, DC, 20005, US

(73) Assignee: Boston Scientific Scimed, Inc.

(21) Appl. No.: 11/140,993

(22) Filed: Jun. 1, 2005

Related U.S. Application Data
(62) Division of application No. 10/106,184, filed on Mar. 27, 2002.

(51) Int. Cl. A61M 29/00

(52) U.S. Cl. 606/192

(57) ABSTRACT

A curved-wing balloon is provided herein. A medical device embodying the invention may include an expandable balloon and an inner shaft positioned within the expandable balloon wherein the expandable balloon may be coupled to an inflation lumen, expandable from a first position to a second position, and having non-parallel sides when initially formed. A method employing the present invention may include placing an expandable medical balloon between a first forming blade and a second forming blade of a cam-former jaw system, moving the first forming blade from a first open position to a second closed position and moving the second forming blade from a first open position to a second closed position, the first forming blade having a first forming member, the second forming blade having a second forming member, the first forming member and the second forming member being in contact with the expandable balloon when in the second closed position, a mating surface of the first forming member being non-parallel to a mating surface of the second forming member when in contact with the balloon.

### FIELD OF THE INVENTION

The present invention relates to methods, systems, and apparatus for deploying expandable medical implants. More particularly the present invention regards methods, systems, and apparatus that employ a curved wing balloon for deploying a medical implant at a target site.

### BACKGROUND

Expandable medical implants are positioned and placed in the body during the completion of numerous contemporary medical procedures. These implants may be used for innumerable purposes including physically reinforcing damaged vessels, replacing ruptured vessels, and delivering therapeutic to a target site in the body.

These medical implants, which can include stents, are often delivered to their target site by an expandable balloon typically located at the distal end of a catheter. In use, when this balloon is positioned at the target site, a medical practitioner will direct fluid into the balloon to inflate the balloon and expand the implant. Then, once the implant has reached a desired size, it will be deployed from the balloon and the balloon will be removed from the target site.

For numerous reasons these balloons are often folded so that they form wings or protrusions that unfold as they expand. While these wings or protrusions can provide the benefits of increased inflation speed and larger balloon size they can also impose unwanted uneven torsional forces on the implant during expansion thereby ripping or tearing away coatings resident on the implant. Both the coating damage and the twisting are undesirable because they can each compromise the effectiveness of the implant in its final deployed state.

Various techniques for creating wings or protrusions and for folding the balloons are available. These techniques generally employ a forming stage and a tipping or folding stage, the forming stage involving the creation of one or more wings on the balloon and the tipping or folding stage involves tipping these protrusions over to wrap them around the balloon.

Automatic cam-formers are available to form the protrusions, or wings, while the folding or tipping process is often a manual one, completed piecemeal by an operator. Hand-folding can be cumbersome and untenable as it requires a high degree of manual dexterity and is susceptible to randomly introducing foreign matter onto the surface of the balloon. Moreover, hand-folding is also inconsistent as its results vary from operator to operator.

### SUMMARY OF THE INVENTION

A curved-wing balloon is provided herein. A medical device embodying the invention may include an expandable balloon and an inner shaft positioned within the expandable balloon wherein the expandable balloon may be coupled to an inflation lumen, expandable from a first position to a second position, and having non-parallel sides when initially formed.

A method employing the present invention may include placing an expandable medical balloon between a first forming blade and a second forming blade of a cam-former jaw system, moving the first forming blade from a first open position to a second closed position and moving the second forming blade from a first open position to a second closed position, the first forming blade having a first forming member, the second forming blade having a second forming member, the first forming member and the second forming member being in contact with the expandable balloon when in the second closed position, a mating surface of the first forming member being non-parallel to a mating surface of the second forming member when in contact with the balloon.

### BRIEF DESCRIPTION OF THE DRAWINGS

FIG. 1 is a cross-sectional view of an inflatable curved-wing balloon in accord with an embodiment of the present invention.

FIG. 2 is a cross-sectional view of the curved-wing balloon of FIG. 1 during inflation.

FIG. 3 is a cross-sectional view of the curved-wing balloon of FIG. 1 also during inflation.

FIG. 4 is a cross-sectional view of the curved-wing balloon of FIG. 1 in a fully inflated condition.

FIGS. 5 a-d are sequential views of a process of forming a curved-wing balloon, in a cam-former, in accord with another embodiment of the present invention.

FIG. 6 is a perspective view of a curved-wing balloon being drawn through a tipping or folding die and into a medical implant in accord with another alternative embodiment of the present invention

FIG. 7 is a perspective view of a folded, curved-wing balloon inside a medical implant in accord with another alternative embodiment of the present invention.

FIG. 8 shows a curved-wing balloon in a stent after the balloon has been fully expanded in accord with an alternative embodiment of the present invention.

### DETAILED DESCRIPTION

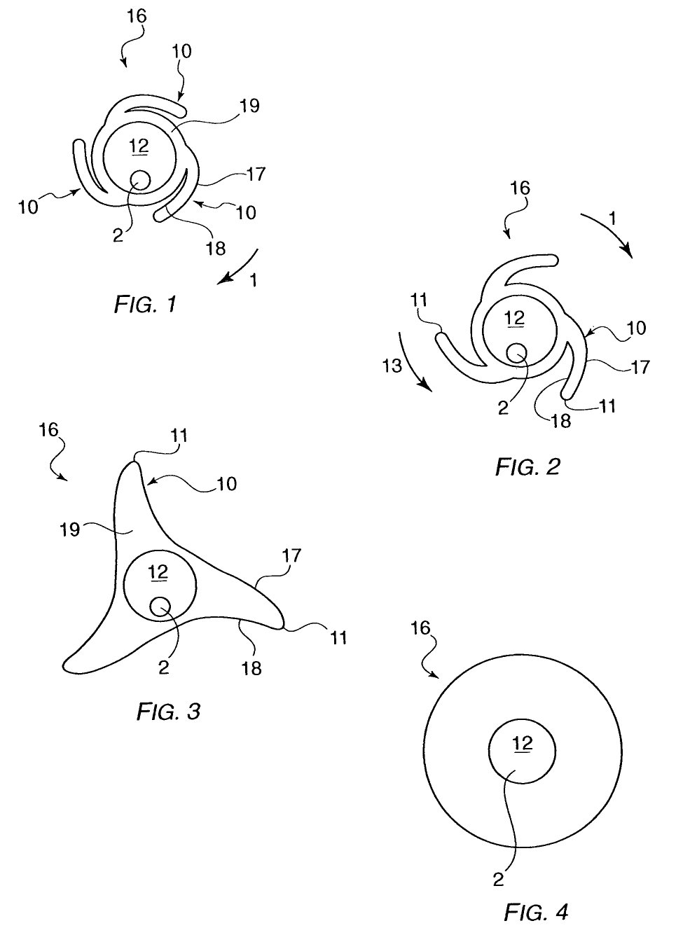

FIGS. 1-4 are cross-sectional views of an expandable curved-wing balloon, mounted on the distal end of a catheter, in various states of inflation in accord with an embodiment of the present invention. In this embodiment, the wings, or protrusions, 10 of the balloon 16 have been formed with a curved cross-sectional profile so that they may be more readily placed at or folded over to a non-radial angle from the central lumen as they are folded around the balloon 16. The curved shape of the protrusions 10 may be imposed on them during their initial forming and at other suitable occasions during the manufacture and subsequent handling of the balloon 16. In each of these circumstances, though, it is preferable that the curvature be added prior to the folding of the wings 10 around the balloon's central portion 19.

In one embodiment discussed in detail below, a cam-former is used to form the curved wings 10 of the balloon and an elongated tipping die is used to fold the wings 10 over around the balloon 16.

FIG. 1 shows a curved-wing balloon 16 in an uninflated condition in accord with an embodiment of the present invention. The curved-wing balloon 16, which surrounds inner shaft 12 and internal lumen 2, has a plurality of wings 10 that extend from its central portion 19. The wings 10 in this embodiment have curved inner and outer walls 17 and 18. These curved inner and outer walls 17 and 18 follow the same approximate rate of curvature as the central portion 19 of the balloon 16 in this embodiment, however, in other embodiments, the rate of curvature may be different—perhaps being more or less curved than the body of the balloon 19. The balloon 16 in FIG. 1 is shown in an uninflated state.

FIG. 2 shows a partially inflated cross-section of the balloon 16 from FIG. 1. As can be seen in FIG. 2, the wings 10 of the balloon 16 begin to unwind in the direction of arrow 13 as fluid is injected into the balloon 16 from the lumen 2 to inflate the balloon. Thus, as can be seen in FIG. 2, the curved orientation of protrusions 10 not only provide them with a propensity to tip in the direction of arrow 1 when the balloon 16 is being folded but the curvature of the wings 10 may also influence the shape of the wings as they unfold in the direction of arrow 13.

FIG. 3 shows the balloon from FIG. 2 after the protrusions 10 have been more fully inflated and have reached their fully extended position. In other words the tips of each of the wings are at their furthest point from the center of the balloon in FIG. 3.

In FIG. 3, the predisposed curvature of the inner and outer walls 17 and 18 of the protrusions is prevalent. Moreover, as can be seen, as the wings 10 of this embodiment expand, rather than protruding linearly out from the central portion 19 of the balloon 16, the protrusions have, instead, retained their curved orientation. As is also evident in FIG. 3, due to their uniquely formed shape, as the wings 10 inflate, their cross-sections may have a visible taper with the portion of the protrusion 10 closer to the central portion 19 of the balloon being wider than the rounded tip end 11 of the wing 10.

FIG. 4 is also a cross-sectional view of balloon 16. In FIG. 4, balloon 16 is shown in its fully expanded state with the inner shaft 12 and internal lumen 2 being clearly shown in the center of the balloon 16. However, while this inner shaft 12 and lumen 2 are shown in the center of the balloon in this embodiment they may also, in other embodiments, be located in other positions relative to the outside wall of the balloon.

While a three wing balloon is provided and described in the embodiment of FIGS. 1-4, in alternative embodiments more or less protrusions may be used. Furthermore, the number of wings employed may depend upon the individual circumstances of the implant being deployed, the coating that may be resident on the implant, and any other number of factors. For instance, if the coating of the implant were susceptible to being rubbed off, the number of protrusions may be increased to reduce the amount of force placed on the coating by each of the wings 10 during expansion. Likewise, if the implant being deployed was easily damaged by concentrated forces placed thereon the number of protrusions could be increased to reduce the forces that it will place on the implant.

Furthermore, while the wings 10 are illustrated as expanding in a clockwise direction in FIGS. nos. 1-4 they may also inflate in a counter-clockwise direction. Still further, in addition to being entirely concentric with the central portion 19 of the balloon 16, a portion of the protrusions 10 may be curved outwardly, opposite the direction of curvature of the central portion 19 to accommodate a specific implant, a specific coating or some other specific design criteria.

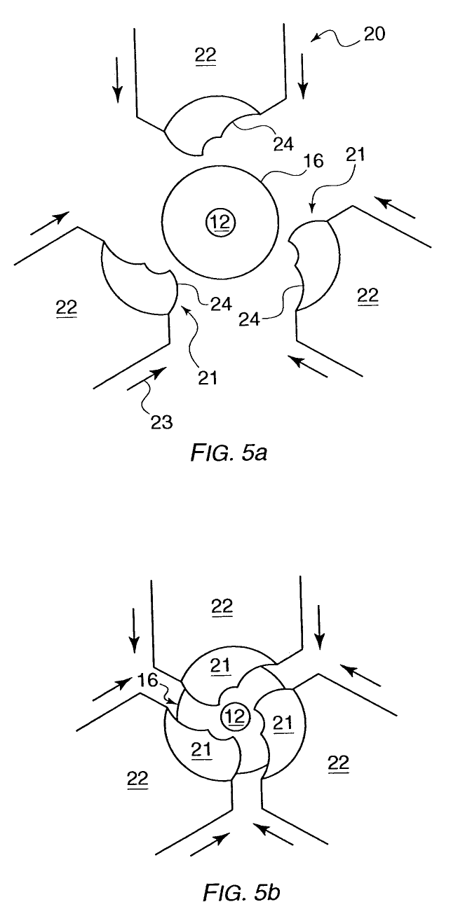

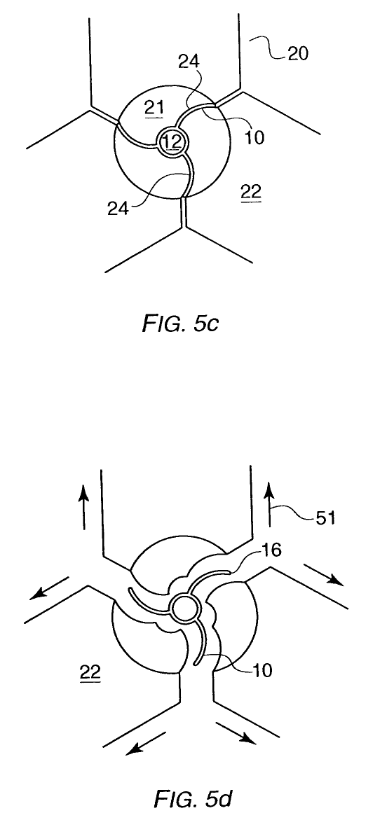

FIGS. 5a-5d are a series of illustrations showing the form stamping of a curved-wing balloon in accord with an alternative embodiment of the present invention. In FIG. 5a an enlarged cross-sectional view of the forming blades 22 of a cam-former 20 are shown. As can be seen the forming blades 22 of the cam-former each has a profiled member 21 with curved mating faces 24.

In use, and as indicated by arrows 23, these forming blades 22 slide within the cam-former 20 and may be used to form protrusions in a balloon 16. Due to the shape of the profiled members 21 and mating faces 24, three curved wings will be formed on the balloon 16

FIG. 5b shows an initial step in the stamping or forming process of the balloon 16. In FIG. 5b, the forming blades 22 are shown merging in on one another and interfacing with the balloon 16 to form the curved protrusions illustrated in FIGS. 1-4.

FIG. 5c shows the forming blades 22 and their mating faces 24 of the cam-former 20 in their inner most position. The curved orientation of the mating faces of the profiled members 21 and the curved orientation of the wings 10 can be readily seen in this figure. During this step, as well as during others, heat and other formative influences may be used to facilitate the formation of the curved-wings 10. In other words, the members 21 and their mating faces 24 may be heated to assist in setting the protrusions 10 during the stamping process. Alternatively, the members 21 may be cooled or various setting compounds may be interfaced with the balloon to set the curvature in the wings 10 during the stamping process. In each of these alternative embodiments, it is nevertheless preferred that some bias be introduced into the shape of the protrusions 10 so that the balloon wings do not become folds with parallel sides protruding orthogonally from the central axis of the balloon.

FIG. 5d illustrates the members 21 moving away from each other in the direction of arrows 51 after the balloon 16 has been stamped and the protrusions 10 have been formed. As can be seen, after the stamping, the wings 10 of the balloon 16 have retained a curved non-parallel and non-planar profile. A profile that may facilitate the later wrapping or tipping of the protrusions around the balloon 16.

In this embodiment, the shape of the profiled member 21 and the mating faces 24 determined the profile of the wings 10. As can be seen, this profile has an arcuate shape within the protrusions extending from the center of the balloon in a clockwise direction. As described below, this “pre-folded” form facilitates the actual folding or tipping of the wings, during later stages of manufacture.

In an alternate embodiment, the balloon could be formed so that the protrusions are curved in a counter-clockwise direction. Likewise, different degrees of curvature may be chosen for different applications. Furthermore, during the manufacturing process when the wings 10 are formed and mating faces 24 are still pressed against the balloon 16, a positive pressure may be applied to the folds while the blades 22 are retracted as shown in FIG. 5d.

The forming blades 22 of the cam-former 20 may be made from any suitable rigid material and may be controlled by any suitable activation mechanism. It is preferred that the mating faces 24 be smooth and that their surfaces have non-adhesive properties so that they do not stick to the balloon 16 during stamping. Moreover, the forming blades 22 may be brought together for varying periods of time and under various pressures depending upon the properties of the balloon being stamped. Likewise, the distance between the mating faces 24 may be increased or decreased depending upon the thickness or other properties of the balloon being stamped.

Once the balloon 16 has been stamped the protrusions 10 may be tipped back onto the balloon to prepare the balloon to receive the implant that it will carry.

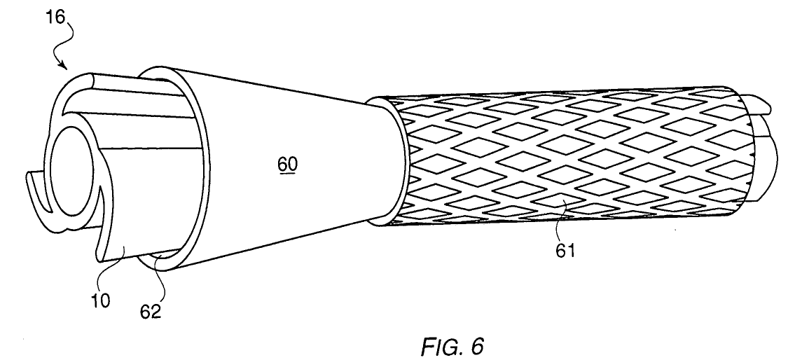

FIG. 6 shows a curved-wing balloon and a tipping die 60 in accord with an alternative embodiment of the present invention. In FIG. 6 a previously stamped curved-wing balloon 16 is shown being drawn through a tipping die 60 and into a medical implant 61. As can be seen in FIG. 6, as the previously folded balloon is moved through the die 60, due to the natural bias of the folds 10, they may fold down upon themselves as the balloon 16 travels though the narrowing internal channel 62 of the die 60. The die 60 in this embodiment may be heated while the balloon is being drawn through it to further heat-set the tipped protrusions of the balloon 16. Alternatively, other external forces such as a vacuum and a positive pressure may also be placed on the balloon 16 to assist in tipping the wings 10. For instance, a positive pressure may be applied between the internal channel 62 and the balloon 16 to allow for the proper tipping or wrapping of the protrusions 10 while a vacuum may be applied within the balloon 16 to draw the wings in as they pass through the die 60.

As can be seen in FIG. 6, the speed of the deployment process can be further increased, by positioning a medical implant 61 at the narrow end of the die 60 so that the tipped balloon is immediately drawn into the implant at the end of the tipping process. Once the implant has been placed over the tipped or folded balloon, it may then be crimped onto it. Alternatively, the balloon 16 may be moved through the die 60 and the implant may be placed onto it at a later date.

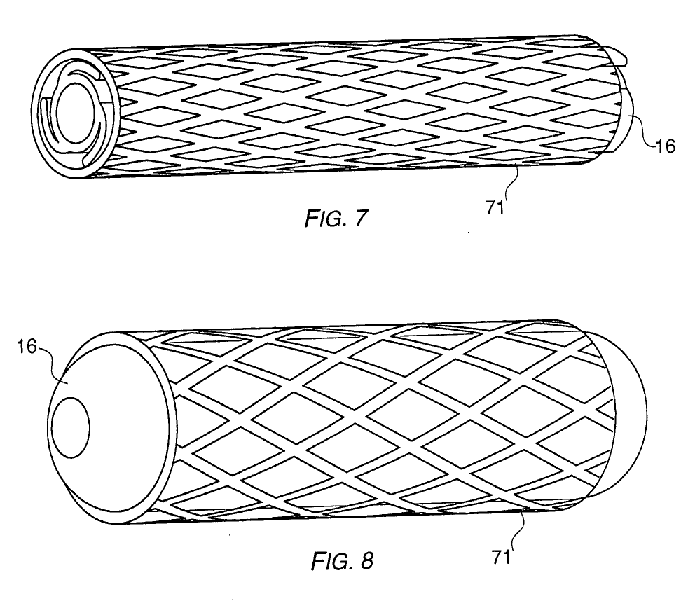

FIG. 7 is a schematic of a curved-wing balloon 16 after it has been placed inside and crimped to a stent 71 in accord with an alternative embodiment of the present invention.

FIG. 8 shows a curved-wing balloon 16 after it has been placed inside a stent 71 and fully expanded.

The implant used in these various embodiments may be any one of numerous medical implants including a stent, an aneurism coil, a vena-cava filter, an a/v shunt, and a stent-graft. In some embodiments the implant may be coated with any one of the various available coatings. This coating may be used to carry or transport therapeutic, to facilitate the acceptance of the implant at the target site, to facilitate the rehabilitation of the target site, and to simply lubricate the folds as they unwind during inflation.

While various embodiments of the present invention are disclosed above other embodiments are also plausible without straying from the spirit and scope of the present invention. For instance, while the tipping die is shown as a solid structure it may, instead, have a lattice wall thereby allowing various gases to be blown onto the balloon during the tipping and folding process.

1. An intra-lumenal medical device comprising:
an expandable balloon having an outside surface; and
an inner shaft positioned within the expandable balloon,
the expandable balloon being coupled to an inflation lumen,
the expandable balloon expandable from a first position to a second position,
the expandable balloon having at least one protrusion formed in the outside surface, and
the protrusion having nonparallel sides when initially formed.

2. The medical device of claim 1 wherein the protrusion has a base in communication with said outside surface and said nonparallel sides, and the protrusion has an extremity in communication with said nonparallel sides.

3. The medical device of claim 1 wherein the protrusion is formed with a forming die so that the sides extend radially from the outside surface with a forming die.

4. The medical device of claim 1 wherein the protrusion is formed with a forming die so that the sides extend tangentially from the outside surface.

5. The medical device of claim 1 wherein the protrusion is formed with a forming die so that the sides extend at an angle between 0 and 90 degrees from the outside surface.

6. The medical device of claim 1 wherein the protrusion is wrapped around the outside surface of the balloon and wherein the protrusion is heat-set around said surface.

7. The medical device of claim 1 further comprising a deployable implant positioned around the balloon.

8. The medical device of claim 7 wherein the implant is a stent.

9. The medical device of claim 7 wherein the sides of the protrusion when initially formed are curved.

10. An expandable balloon for expanding a medical implant comprising:
an inner shaft within the expandable balloon;
an internal lumen; and
a protrusion extending from the expandable balloon,
the protrusion having a non-rectilinear profile when initially formed,
the protrusion in fluid communication with the internal lumen.

11. The balloon of claim 10 wherein the center line between a first side of the protrusion and a second side of the protrusion when initially formed is bent.

12. The balloon of claim 10 wherein the protrusion extends from the expandable balloon between radially and tangentially.

13. A medical implant delivery system comprising:
an expandable medical implant; and
an expandable balloon catheter having an expandable balloon at its distal end,
the expanded balloon having a plurality of protrusions,
the protrusions having a non-rectilinear profile when initially formed,
the protrusions wrapped around the expandable balloon and located within the implant.

14. A method of creating protrusions in an expandable medical balloon comprising:
placing an expandable medical balloon between a first forming blade and a second forming blade of a cam-former jaw system;
moving the first forming blade from a first open position to a second closed position; and
moving the second forming blade from a first open position to a second closed position,
the first forming blade having a first forming member,
the second forming blade having a second forming member,
the first forming member and the second forming member being in contact with the expandable balloon when in the second closed position,
a mating surface of the first forming member being nonparallel to a mating surface of the second forming member when in contact with the balloon.

15. The method of claim 14 further comprising:
wrapping a curved protrusion of the expandable balloon around the balloon.

16. The method of claim 14 further comprising:
moving the expandable balloon through a tipping die.

17. The method of claim 16 wherein the tipping die is in the shape of a truncated cone.

18. The method of claim 16 further comprising:
heating the tipping die.

19. A method of making a medical expandable balloon comprising:
inflating a balloon;
radially pressing profiled members having curved interfaces into the inflated balloon to make curved wings; and
wrapping the curved wings around the balloon.

20. The method of claim 19 wherein the wrapping further comprises:
passing the lumen through a tapered tipping die from a wider end to a narrower end.

21. The method of claim 20 wherein the tipping die is heated.

22. The method of claim 20 further comprising moving the expandable balloon into an expandable medical implant.

23. The method of claim 22 further comprising crimping the medical implant onto the balloon.

24. A method of mounting an implant on an expandable balloon catheter comprising:
reshaping an expandable balloon to have a wing, the wing having a curved profile when fully extended;
drawing the expandable balloon through a folding die; and
mounting an implant on the expandable wing balloon.

25. The method of claim 24 further comprising:
heating the folding die.

26. The method of claim 24 further comprising:
crimping the implant onto the expandable balloon.
